(19) United States
(12) Patent Application Publication
(10) Pub. No.: US 2013/0211232 A1
(43) Pub. Date: Aug. 15, 2013

(54) Arthroscopic Surgical Planning and Execution with 3D Imaging

(71) Applicant: The Johns Hopkins University, (US)

(72) Inventors: Ryan J. Murphy, Columbia, MD (US); Mehran Armand, Maple Lawn, MD (US); Marc Hungerford, Cokeysville, MD (US); Yoshito Otake, Baltimore, MD (US); Jyri Lepisto, Tampere (FI)

(73) Assignee: THE JOHNS HOPKINS UNIVERSITY, Baltimore, MD (US)

(21) Appl. No.: 13/756,825

(22) Filed: Feb. 1, 2013

Related U.S. Application Data
(60) Provisional application No. 61/593,655, filed on Feb. 1, 2012.

(51) Int. Cl. A61B 5/00; A61B 6/03; A61B 17/32; A61B 1/04; A61B 1/00; A61B 5/055; A61B 1/317

(52) U.S. Cl. CPC A61B 5/4836; A61B 5/0077; A61B 5/055; A61B 6/032; A61B 5/0035; A61B 1/317; A61B 1/04; A61B 1/00045; A61B 1/00009; A61B 1/00087; A61B 17/320016
USPC 600/411; 600/476; 600/427; 600/109; 600/104

(57) ABSTRACT

A method includes obtaining a first three-dimensional (3-D) image of a bone structure, generating a surgical plan based on the first 3-D image and registering the surgical plan to the bone structure to generate a registered surgical plan by obtaining a first 2-D real-time video image of the bone structure and a second 3-D image of the bone structure, and correlating structures from the first 2-D real-time video image and the second 3-D image with the surgical plan. The method also includes obtaining a second 2-D real-time image of the bone structure and overlaying the registered surgical plan onto the second 2-D real-time video image.

### CROSS-REFERENCE TO RELATED APPLICATIONS

This application claims priority to and the benefit of prior-filed co-pending U.S. Provisional Application No. 61/593,655, filed Feb. 1, 2012, the content of which is herein incorporated by reference in its entirety.

### STATEMENT OF GOVERNMENTAL INTEREST

This invention was made with government support under contract number R01EB006839 awarded by the National Institutes of Health (NIH). The government has certain rights in the invention.

### BACKGROUND

Example embodiments of the present invention relate to arthroscopic surgery, and in particular to the planning and execution of arthroscopic surgery using three-dimensional imaging.

During sports activities, other strenuous activities and even daily activities, damage may occur to joints to due to recurring irritation of the joints. Patients with joint damage experience pain and limited range of motion. Some joints are easy to access, but other joints, such as the hip joint, may be relatively difficult to access and diagnose.

Femoroacetabular impingement (FAI), which is a major cause of early osteoarthritis of the hip, is characterized by early pathologic contact during hip joint motion between skeletal prominences of the acetabulum and the femur that limits the physiologic hip range of motion. Radiographs, which are commonly used to estimate an amount of resection during surgery, may suffer from inaccuracies. For example, in pincer-type impingement, pelvic tilt and rotation changes the amount, or apparent existence, of crossover in patients, where the crossover corresponds to the portion of the anterior acetabular rim that projects laterally past the posterior rim in a standard pelvis radiograph.

In addition, because of the limited viewing range and image distortion during arthroscopic surgery, accurate execution of a planned bone resection may be difficult.

### SUMMARY

A method according to one embodiment of the present invention includes obtaining a first three-dimensional (3-D) image of a bone structure, generating a surgical plan based on the first 3-D image and registering the surgical plan to the bone structure to generate a registered surgical plan by obtaining a first 2-D real-time video image of the bone structure and a second 3-D image of the bone structure, and correlating structures from the first 2-D real-time video image and the second 3-D image with the surgical plan. The method also includes obtaining a second 2-D real-time image of the bone structure and overlaying the registered surgical plan onto the second 2-D real-time video image.

A surgical system according to one embodiment of the present invention includes an arthroscopic camera configured to obtain a first real-time image of a bone structure at a first time and a second real-time image of the bone structure at a second time and a first three-dimensional (3-D) imaging apparatus configured to generate 3-D data corresponding to the bone structure. The system also includes a registration unit configured to register a stored surgical plan with the bone structure based on the first real-time image of the bone structure and the 3-D data to generate a registered surgical plan. The system also includes a composite image generator configured to overlay onto the second real-time image data from the registered surgical plan to generate a composite image and a display device configured to display the composite image.

A surgical system according to one embodiment of the present invention includes an arthroscopic camera configured to obtain a real-time image of a bone structure and a composite image generator configured to overlay onto the real-time image data from a stored surgical plan. The system also includes a display device configured to display the composite image.

Additional features and advantages are realized through the techniques of the example embodiments of the present invention. Other embodiments are described in detail herein and are considered a part of the claimed invention. For a better understanding of the invention with the advantages and the features, refer to the description and to the drawings.

### BRIEF DESCRIPTION OF THE SEVERAL VIEWS OF THE DRAWINGS

The subject matter which is regarded as the invention is particularly pointed out and distinctly claimed in the claims at the conclusion of the specification. The forgoing and other features, and advantages of embodiments of the invention are apparent from the following detailed description taken in conjunction with the accompanying drawings in which:

FIG. 1 illustrates a method of generating and executing a surgical plan according to an embodiment of the invention;

FIG. 2 illustrates a diagnostic system according to an embodiment of the invention;

FIG. 3 illustrates a registration system according to an embodiment of the present invention; and

FIG. 4 illustrates a surgical system according to an embodiment of the present invention.

### DETAILED DESCRIPTION

Surgical procedures, and in particular, arthroscopic surgical procedures, may suffer from inaccurate or incomplete surgical plans and limited viewing range and distortion during a surgical procedure. Embodiments of the invention relate to generating an arthroscopic surgical plan and overlaying the arthroscopic surgical plan onto a real-time image during a surgical procedure.

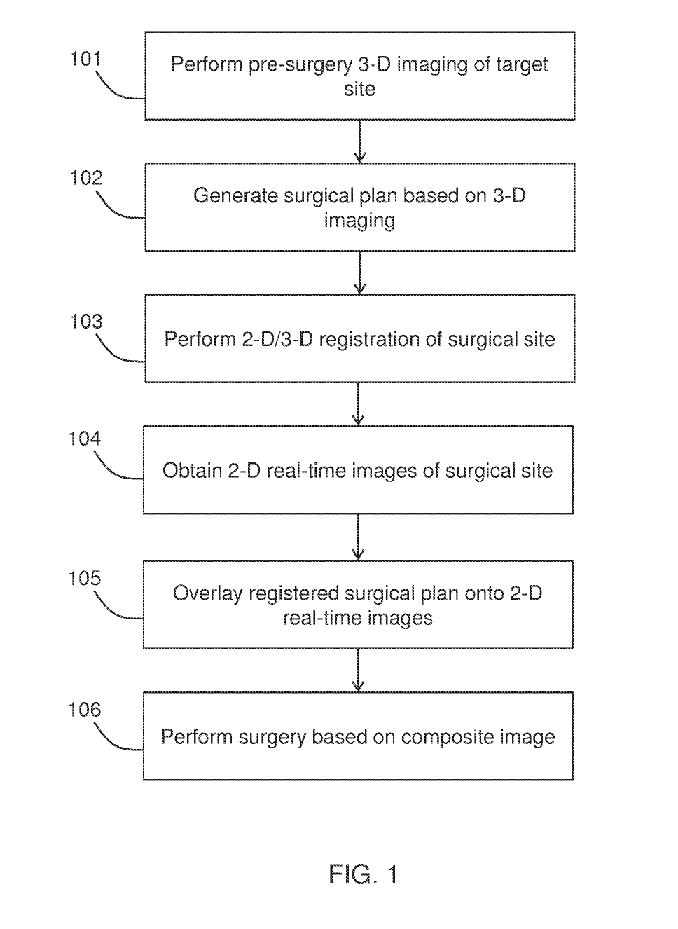

FIG. 1 illustrates a flow diagram of a method according to an embodiment of the invention. In block 101, a three-dimensional (3-D) imaging of a target site. For example, in one embodiment a 3-D image is generated by placing a patient or a portion of a patient in a magnetic resonance imaging (MRI) device. In another embodiment, the 3-D image is generated based on a combination of an MRI image with an x-ray computed tomography (CT) scan. In one embodiment, the target site is a bone structure, such as a joint. In one embodiment, the joint is a hip joint formed by a socket of a pelvis and a cam of a femur. The 3-D image of the bone structure may be configured to measure the cross-over of the bone structure, which is defined as the degree to which the anterior rim of the acetabulum projects laterally past the posterior rim.

In block 102, a surgical plan is generated based on the 3-D image generated from the 3-D imaging. The surgical plan may involve any cutting or resection of bone based on characteristics detected in the 3-D image. In one embodiment, the characteristics of the 3-D image are compared to reference characteristics to identify portions of the bone structure that are candidates for surgery. For example, in an embodiment in which the bone structure is a hip joint, the identified characteristics may correspond to a bump on a femur or an impingement of a pincer resulting from acetabular overgrowth.

The surgical plan may be a digital file that, when executed, identifies in three dimensions portions of the bone structure that are to be subject to surgical treatment. In one embodiment, the surgical plan may be displayed as a 3-D image. In one embodiment, the surgical plan is automatically generated by a computer based on the computer receiving the 3-D image from the 3-D imaging device and the computer comparing the 3-D image with reference data. In another embodiment, the surgical plan is generated based on at least some user input. For example, an operator may view the 3-D image on a display device, and the 3-D image may be overlaid with reference data to identify regions that may be targeted for surgery. The user may then manually select or identify portions of the bone structure that will be targeted for surgery in a subsequent surgical procedure.

In block 103, registration of the target site is performed to register the 3-D surgical plan and surgical tools with respect to the bone structure of the patient. In embodiments of the present invention, the registration is performed with two or more imaging devices including an arthroscope to obtain a 2-D video image and one or more of an x-ray imaging device to obtain x-ray images, an optical tracker to obtain tracking data or an electromagnetic tracking device to obtain imaging data. The x-ray images, optical tracking and electromagnetic tracking devices provide 3-D data of the bone structure, arthroscope and any surgical instruments. During registration and surgery, real-time images of a surgical site are obtained. For example, an incision may be made and an arthroscope may be inserted into the incision and maneuvered to the surgical site to capture real-time images of the surgical site, corresponding to the target site of the surgical plan. In one embodiment, the real-time images may be two-dimensional (2-D) images. For example, the arthroscope may include a video camera to capture real-time video images of the surgical site.

The registration of the target site maps the actual surgical site to the 2-D and 3-D data. In embodiments of the invention, the registration is performed without leaving the physical tags or markers on the bone structures during surgery. Instead, registration may be performed only with imaging devices, such as the arthroscope and optical tracker, x-ray device, or electromagnetic imaging device, as discussed above.

In block 104, 2-D real-time images of the surgical site are again obtained, for example, by the arthroscope.

In block 105, the location information obtained during registration of the surgical site is used to overlay the registered surgical plan, or data generated from the surgical plan, onto the real-time images generated by the arthroscope to generate a composite image. The overlaying of the surgical plan onto the real-time images may include overlaying colors onto different portions of the bone structure to identify the different portions. For example, a portion of the 2-D video image corresponding to the pelvis may be overlaid with a first color and a portion corresponding to the femur may be overlaid with another color. The different portions of the bone structure in the 2-D real-time image may be identified based on the 3-D surgical plan. In addition, particular sites that are targets for surgery may be designated by the overlaying of the surgical plan, or data generated by the surgical plan, onto the real-time images. For example, a bump on a cam of a femur or an overgrowth on a pincer of a pelvis displayed in the 2-D real-time images may be overlaid with a predetermined color to identify the portion of the bone structure as being a target for surgery.

In one embodiment, overlaying the surgical plan, or a portion of the surgical plan, onto the 2-D real-time image includes identifying a location and inclination of an arthroscope. In another embodiment, overlaying the surgical plan, or a portion of the surgical plan, onto the 2-D real-time image includes identifying similarities between portions of the bone structure shown in the 2-D real-time images and the 3-D surgical plan.

In block 106, surgery is performed based on the composite image. For example, the composite image may be displayed on a display device provided to a surgeon. In one embodiment, as the surgery is performed, the data from the surgical plan is updated based on the real-time images. For example, if a point-of-view of the real-time image changes, the data from the surgical plan overlaid onto the real-time image also changes to correspond to the changed point-of-view. In another embodiment, if portions of the bone structure are removed in the surgery, the surgical plan is changed to reflect the changed shape of the bone structure. Accordingly, a surgeon may visually see on the display when targeted portions of the bone structure have been removed. In addition, in embodiments where real-time three-dimensional data is generated, such as by an optical tracker or an electromagnetic tracker, the location of surgical tools may be tracked and included in the composite image.

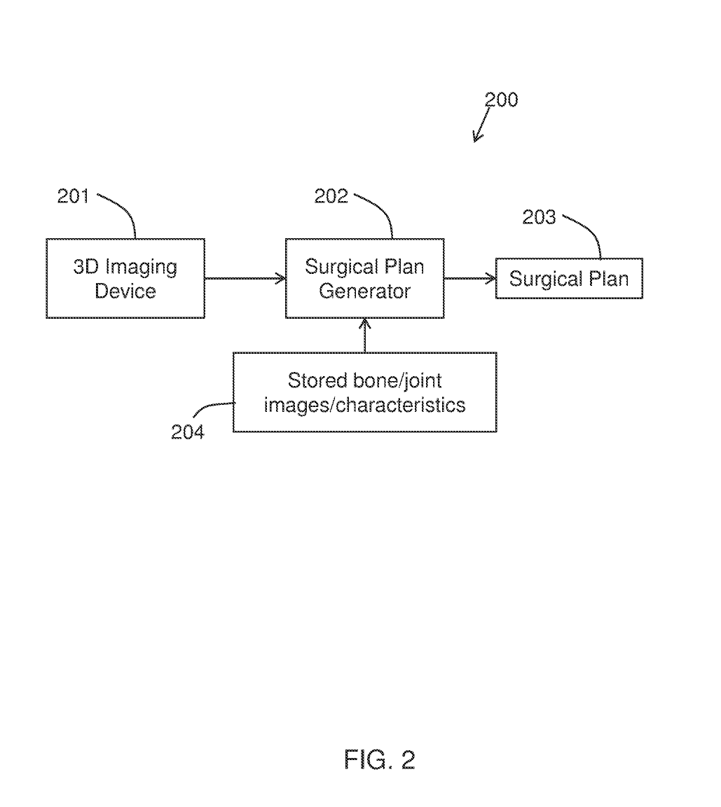

FIG. 2 illustrates a diagnostic plan system 200 according to an embodiment of the invention. The system 200 includes a 3-D imaging device 201, such as an MRI device. The 3-D imaging device 201 obtains a 3-D image of a portion of a body, such as a bone structure in a body and transmits the 3-D image to a surgical plan generator 202. In one embodiment, the bone structure is a joint, such as a hip joint. The surgical plan generator 202 compares the 3-D image with stored bone or joint images or characteristics. The images or characteristics 204 may identify ideal or typical bone structures or relationships between bone structures. The images or characteristics 204 may also identify abnormal bone structures. For example, in an embodiment in which the 3-D image is of a hip joint, the stored images or characteristics 204 may identify a range of characteristics of femoral heads and pelvic sockets that are considered normal, and the surgical plan generator 202 may identify targets for surgery by comparing the reference images or characteristics with the 3-D image obtained by the 3-D imaging device 201. In another embodiment, the stored images or characteristics 204 identify typical characteristics of bone disease or defect, and the surgical plan generator 202 generates a surgical plan based on detecting similarities between the images or characteristics 204 and the 3-D image obtained by the 3D imaging device 201.

In one embodiment, the surgical plan generator 202 is a computer including processing circuitry to analyze and compare a 3-D image and stored images or characteristics. In one embodiment, the surgical plan generator 202 includes a display device to display one or both of the 3-D image obtained by the 3-D imaging device 201 and the stored images or characteristics 204. In such an embodiment, a user may analyze the 3-D image, or an overlay of the stored image or characteristics onto the 3-D image, to select portions of the 3-D image that are candidates for surgery. Based on one or both of the comparisons performed by the computer and the user input, a surgical plan 203 is generated by the surgical plan generator 202.

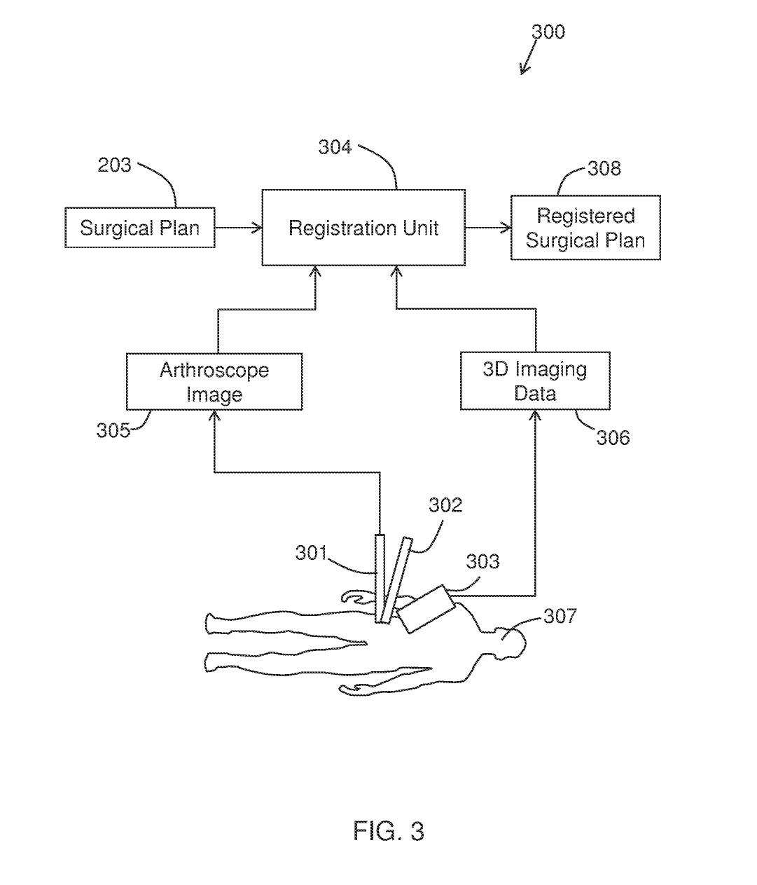

FIG. 3 illustrates a registration system 300 according to an embodiment of the invention. The system 300 includes an arthroscope 301, surgical tool 302 and another imaging device 303, such as an optical tracker, and a registration unit 304. The arthroscope 301 is inserted into an incision in a patient 307 to obtain a 2-D video image 305 of a surgical site, and in particular of a bone structure of a surgical site. The imaging device 303 may be a 3-D imaging device 303 that tracks the location of the arthroscope 301, surgical tool 302 and features of the surgical site, such as the bone structure of the surgical site to generate 3D imaging data 306. Examples of 3-D imaging devices include an optical tracker which provides 3-D data, an x-ray device that generates x-ray images, and an electromagnetic tracker that generates 3-D data.

Based on the imaging information obtained by the arthroscope image 303 and the 3-D imaging data 306, the registration unit 304 registers the surgical plan 203 and the surgical tool 302 with respect to the surgical site of the patient 307 to obtain a registered surgical plan 308.

Registration of the surgical site may be performed using both the 2-D arthroscopic image and a 3-D image, and the 2-D and 3-D images may be used together to register the surgical plan 203 and surgical tool 302 with respect to the patient 307. Accordingly, in embodiments of the invention, it is not necessary to physically contact the surgical site to perform registration or to leave physical tags on structures of the surgical site for registration.

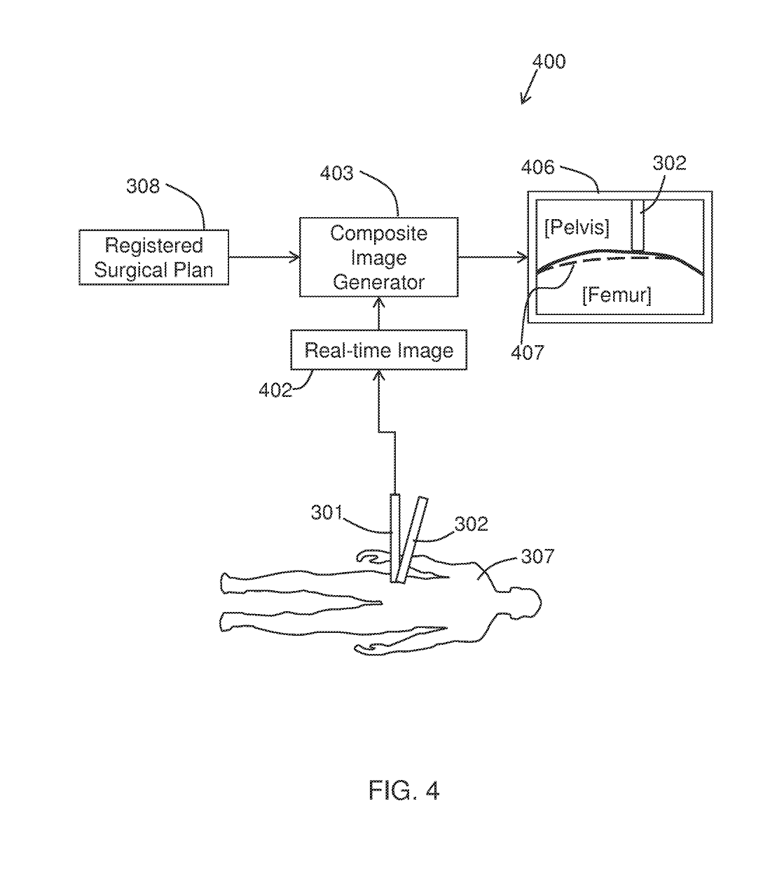

FIG. 4 illustrates a surgical system 400 according to an embodiment of the invention. The system 400 includes an arthroscope 301 configured to generate real-time images 402 of a surgical site, such as a bone structure in a human body 307. The arthroscope 301 may be inserted into an incision prior to, or at the same time as, insertion of one or more surgical tools 302 into an incision. The surgical tool 302 may be, for example, a cutting tool to cut bone from a bone structure.

The composite image generator 403 receives the registered surgical plan 308 from the registration unit 304 and generates a composite image that includes both data from the registered surgical plan 308, or a portion of the registered surgical plan 308, and the real-time image 402. The resulting image is displayed on a display device 406. For example, in FIG. 4, a composite image may illustrate a pelvis from the real-time image 402 in one color and a femur in another color. The pelvis and femur may be identified based on the registered surgical plan 308. In other words, the registered surgical plan 308 may provide a 3-D map of the surfaces of the bones of a bone structure, and as the arthroscope 301 captures images of the surfaces in the real-time images 402, the composite image generator 403 may correlate the portions of the 3-D registered surgical plan 308 that correspond to the structures of the 2-D real-time images 402, and may overlay data from the registered surgical plan 308, such as color-coding data, onto the 2-D real time images 402.

In one embodiment, the composite image generator 403 also overlays onto the real-time images 402 data corresponding to a target region 407 that has been identified as being a target for surgical treatment, such as excess bone that has been targeted for removal. The target region 407 may be overlaid with a different color than non-target regions. For example, referring to FIG. 4, most of the femur may be designated by the color green while the target region 307 of the femur may be designated by the color red. In one embodiment, as bone is removed by the surgical tool 302, the registered surgical plan 308 is updated to correspond to the new surface shapes of the bones of the bone structure.

Although the diagnostic plan system 200 of FIG. 2 and the registration system 300 of FIG. 3 are illustrated in separate figures from the surgical system 400 of FIG. 4, embodiments of the invention encompass a combined system. For example, the surgical plan generator 202, registration unit 304 and composite image generator 403 may be part of the same computer, such as programs executed by one or more processors, or processing circuits housed within the same computer housing, such as the housing of a personal computer or server.

In embodiments of the invention a surgical plan is generated and executed based on a 3-D image of a bone structure. Registration of the surgical plan is performed using a 2-D arthroscopic image and a 3-D image or 3-D data, such as image data from an optical tracker, x-ray images or electromagnetically-generated images. The combined 2-D and 3-D data is used to register the surgical plan and surgical tools with respect to a patient. During execution, data from the 3-D surgical plan and the 2-D/3-D registration (including, for example, surgical tools) is overlaid onto a 2-D arthroscopic image of the surgical site, and the composite image is displayed to help a surgeon perform the surgery. Embodiments of the invention encompass any bone structure, and particularly any joint. Benefits of embodiments of the present invention are particularly realized when a joint is difficult to access, such as a hip joint. Accordingly, an embodiment of the invention will be described in additional detail below with respect to a hip joint and in particular with respect to arthroscopic treatment of pincer-type femoroacetabular impingement with 3-D surgical planning.

In a preoperative procedure, an MRI and/or x-ray computed tomography (CT) scan of a patient may be obtained. 3-D volumetric models of the pelvis and femur are reconstructed based on the MRI and CT scans. A surgical planning generator, such as the generator 202 of FIG. 2, estimates an optimum amount are area of bone resection on the 3-D volumetric model using anatomical measures including 3-D crossover (the area of the anterior rim of the acetabulum projecting laterally past the posterior rim) and alpha angle (the angle between the neck axis of the femur and the axis passing through the center of the femur head and the point where the cortical margin leaves the sphere of the head).

In one embodiment, acetabular over-coverage resulting from pincer deformity is assessed by performing CT scans of a patient's hip region. The acetabular lunate is then segmented and anterior and posterior rims of the acetabular wall are defined. CT scans are digitally reconstructed as digitally reconstructed radiographs (DRRs) to place the pelvis in a neutral position and the segmented acetabular wall is used to identify the amount of rim over-coverage in the neutral position. The mid-acetabular plane is determined and 3-D crossover is defined, corresponding to locations where the anterior rim crosses the mid-acetabular plane. The points of the crossover section are used to automatically compute several 3-D measurements, including crossover length and width.

During an operation, a patient is anesthetized and a surgeon creates entry ports for minimally-invasive hip arthroscopy. In one embodiment, optical markers for navigation and x-ray markers are be attached to the patient's bone and the optical markers may also be attached to the arthroscope and the resection tools. A registration process is performed using an arthroscope and an optical tracker. Embodiments of the invention also include other registration devices, including an x-ray device to generate a series of x-ray images and an electromagnetic device, or any other imaging device. In embodiments of the invention, a tool is not required to contact a bone structure to register a location of the bone structure, and it is not necessary to leave identification markers on the bone structure during surgery. Instead, registration may be performed using a combination of a 2-D arthroscope and one or more 3-D imaging devices, and if markers are used, such as with an optical tracking device or x-ray imaging, the markers may be removed prior to performing surgery.

To guide the surgeon, the preoperative 3-D reconstructed model may be overlaid on the monoscopic, or 2-D, image obtained from the arthroscope, and the planned resection regions may be highlighted. As the surgeon shaves the bone, the 3-D preoperative model may be updated to reflect the new bone shape.

In one embodiment, an intraoperative workstation includes a PC-based interface between a surgeon and other components in a system including an optical or electromagnetic tracker and arthroscopy examination system with the capability of digitally capturing and streaming images to external devices. Reference rigid body markers may be attached to the arthroscope, the surgical tools and to the patient to provide real-time tracking. The workstation produces both tracking data and images from the arthroscopic system to provide image guidance to the surgeon with the 3-D model overlaid onto the arthroscopic video view. The workstation may also provide visualization of the position of the arthroscope with respect to bone structures and the planned resection.

Accordingly, an accurate 3-D model of a surgical site may be generated prior to a surgery, and a composite image of an arthroscopic video and data from the 3-D model is used to aid a surgeon during surgery.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments have been chosen and described in order to best explain the principles of the invention and the practical application, and to enable others of ordinary skill in the art to understand the invention for various embodiments with various modifications as are suited to the particular use contemplated

While the preferred embodiment to the invention had been described, it will be understood that those skilled in the art, both now and in the future, may make various improvements and enhancements which fall within the scope of the claims which follow.

What is claimed is:

1. A method comprising:
obtaining a first three-dimensional (3-D) image of a bone structure;
generating a surgical plan based on the first 3-D image;
registering the surgical plan to the bone structure to generate a registered surgical plan by:
obtaining a first 2-D real-time video image of the bone structure and a second 3-D image of the bone structure, and
correlating structures from the first 2-D real-time video image and the second 3-D image with the surgical plan;
obtaining a second 2-D real-time image of the bone structure; and
overlaying the registered surgical plan onto the second 2-D real-time video image.

2. The method of claim 1, wherein the three-dimensional image is generated by at least one of a magnetic resonance imaging (MRI) device and a computed tomography x-ray device.

3. The method of claim 1, wherein generating the surgical plan includes generating a three-dimensional representation of the bone structure.

4. The method of claim 3, wherein the three-dimensional representation of the bone structure includes data distinguishing a portion of the bone structure identified for removal.

5. The method of claim 1, wherein obtaining the first and second real-time images includes inserting an arthroscope into a body at a location corresponding to the bone structure.

6. The method of claim 1, wherein the second 3-D image is generated using one of an optical tracker and a set of x-ray images.

7. The method of claim 1, wherein overlaying the registered surgical plan onto the second 2-D real-time video image includes displaying data from the registered surgical plan onto corresponding locations of an image of the real-time image, and
changing data from the surgical plan displayed based on changing the real-time image.

8. The method of claim 1, further comprising:
surgically removing a portion of the bone structure;
updating the surgical plan to account for the portion of the bone structure surgically removed; and
updating the data from the surgical plan displayed based on the updating of the surgical plan.

9. The method of claim 1, wherein overlaying the surgical plan onto the real-time image includes overlaying different colors onto different portions of the bone structure of the real-time image to identify different characteristics of the different portions of the bone structure.

10. The method of claim 1, wherein the bone structure is a joint including a cam and socket.

11. The method of claim 1, wherein the bone structure includes a femoroacetabular impingement (FAI).

12. The method of claim 11, wherein the surgical plan includes a visual identifier of the FAI.

13. A surgical system comprising:
an arthroscopic camera configured to obtain a first real-time image of a bone structure at a first time and a second real-time image of the bone structure at a second time;
a first three-dimensional (3-D) imaging apparatus configured to generate 3-D data corresponding to the bone structure;
a registration unit configured to register a stored surgical plan with the bone structure based on the first real-time image of the bone structure and the 3-D data to generate a registered surgical plan;
a composite image generator configured to overlay onto the second real-time image data from the registered surgical plan to generate a composite image; and
a display device configured to display the composite image.

14. The surgical system of claim 13, wherein the first and second real-time images are a two-dimensional (2-D) video images.

15. The surgical system of claim 14, wherein the stored surgical plan is 3-D representation of the bone structure.

16. The surgical system of claim 15, further comprising:
a second 3-D imaging apparatus configured to generate the three-dimensional representation of the bone structure; and
a surgical plan generation device configured to generate a three-dimensional surgical plan for operating on the bone structure and to store the surgical plan,
wherein the surgical plan generation device is configured to compare the three-dimensional representation of the bone structure to a reference representation of the bone structure and to identify a portion of the bone structure to be surgically removed based on the comparison.

17. The surgical system of claim 13, wherein the first 3-D imaging apparatus comprises one of an optical tracker, an x-ray device, and an electromagnetic tracker.

18. The surgical system of claim 13, wherein the composite image generator is configured to overlay onto the bone structure of the real-time image different colors corresponding to different characteristics of different portions of the bone structure.

19. The surgical system of claim 13, further comprising:
a surgical cutting tool for cutting a portion of the bone structure,
wherein the composite image generator is configured to adjust the registered surgical plan displayed on the composite image based on the cutting of the portion of the bone structure by the surgical cutting tool.
